(19) United States
(12) Patent Application Publication
(10) Pub. No.: US 2015/0209290 A1
(43) Pub. Date: Jul. 30, 2015

(54) BILAYERED COMPOSITE TABLET FORMULATION COMPRISING ATORVASTATIN, IRBESARTAN AND MAGNESIUM CARBONATE

(71) Applicant: HANMI PHARM. CO., LTD, Hwaseong-si, Gyeonggi-do (KR)

(72) Inventors: Yong Il Kim, Suwon-si (KR); Jung Hyun Cho, Incheon (KR); Jun Young Choi, Suwon-si (KR); Young Keun Choi, Daegu (KR); Jae Hyun Park, Suwon-si (KR); Jong Soo Woo, Suwon-si (KR)

(73) Assignee: HANMI PHARM. CO., LTD, Hwaseong-si, Gyeonggi-do (KR)

(21) Appl. No.: 14/421,467

(22) PCT Filed: Aug. 30, 2013
(86) PCT No.: PCT/KR2013/007838

§ 371 Date: Feb. 13, 2015

(30) Foreign Application Priority Data
Aug. 31, 2012 (KR) 10-2012-0096477

(51) Int. Cl. A61K 9/20; A61K 31/40; A61K 9/24; A61K 31/4184

(52) U.S. Cl. CPC A61K 9/2009; A61K 31/4184; A61K 31/40; A61K 9/2095; A61K 9/209

(57) ABSTRACT

Disclosed are a bilayered composite tablet formation comprising (a) a first layer comprising irbesartan or a pharmaceutically acceptable salt thereof; and (b) a second layer comprising atorvastatin or a pharmaceutically acceptable salt thereof and magnesium carbonate (MgCO₃) in a weight ratio of 1:4 to 1:5, and a method for preparing the same. Exhibiting excellent dissolution rates and bioavailability, the bilayered composite tablet formulation is useful as a therapeutic for hypertension and hypercholesterolemia.

### FIELD OF THE INVENTION

The present invention relates to a bilayered composite tablet formulation comprising atorvastatin, irbesartan and magnesium carbonate, which is improved in bioavailability and dissolution rate.

### BACKGROUND OF THE INVENTION

Atorvastatin or a pharmaceutically acceptable salt thereof is a selective and competitive HMG-CoA reductase inhibitor. Particularly, atorvastatin calcium, represented by the formula (I) below (IUPAC name: calcium [R—(R*,R*)]-2-(4-fluorophenyl)-β,δ-dihydroxy-5-(1-methylethyl)-3-phenyl-4-[(phenylamino)carbonyl]-1H-pyrrole-1-heptanoate (2:1)), lowers the level of low-density lipoprotein cholesterol in blood, acting as a lipid-lowering agent useful for the treatment of dyslipidemia. In addition, atorvastatin calcium is known to decrease the mortality attributed to cardiovascular diseases, and reduce the chance of stroke in people at risk.

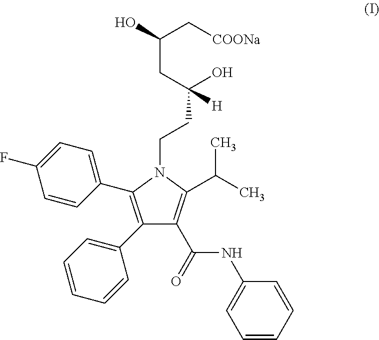

Irbesartan is a potent angiotensin II receptor antagonist, which blocks the interaction of angiotensin II, a causative agent of vasoconstriction, with angiotensin II AT₁ receptors to induce a decrease in blood pressure. Irbesartan selectively inhibits AT₁ receptors, but does not block angiotensin II from binding to AT₂ receptors, thus suppressing endothelial cell growth, vasoconstriction, and tissue regeneration while maintaining the vasodilatation activity.

International Patent Publication No. WO 03/011283 discloses a composite formulation comprising atorvastatin calcium and amlodipine besylate in which an alkalizing agent that forms pH 5 or greater is used as a stabilizer for atorvastatin calcium. In the composite formulation, calcium carbonate, dicalcium phosphate or tricalcium phosphate is employed as an alkalizing agent. Atorvastatin or a pharmaceutically acceptable salt thereof and calcium carbonate are used in a ratio of about 1:1 to 1:4 (w/w). According to the method above, the alkalizing agent guarantees enhanced stability of atorvastatin. In view of the pharmacokinetic or clinical aspect, however, the use of the alkalizing agent requires a larger dose of atorvastatin for a desired therapeutic effect.

Korean Patent Laid-Open Publication No. 2011-126020 describes a bilayered composite tablet formulation composed of a first layer comprising irbesartan or a pharmaceutically acceptable salt thereof, and a second layer comprising an HMG-CoA reductase inhibitor and an alkaline agent, disclosing that the alkaline agent enhances the stability of the HMG-CoA reductase inhibitor and CaCO₃, MgCO₃, or a mixture thereof may be employed as the alkaline agent.

Although such alkaline agents are known to stabilize HMG-CoA reductase inhibitors including atorvastatin, further research is required to improve the dissolution rate or bioavailability of the HMG-CoA reductase inhibitors by using the alkaline agents.

The present inventors have endeavored to improve dissolution rate and bioavailability of a composite formulation comprising irbesartan and atorvastatin; and have found that the coexistence of magnesium carbonate at a specific weight ratio with atorvastatin in a layer exerts an excellent influence on the improvement of the drug in dissolution and uptake into the body, and thus accomplished the present invention.

### SUMMARY OF THE INVENTION

Accordingly, it is an object of the present invention to provide a pharmaceutical composite formulation comprising irbesartan and atorvastatin which exhibits excellent bioavailability and an optimal dissolution profile.

It is another object of the present invention to provide a method for preparing the pharmaceutical composite formulation.

In accordance with one aspect of the present invention, there is provided a bilayered composite tablet formulation comprising:

(a) a first layer comprising irbesartan or a pharmaceutically acceptable salt thereof; and

(b) a second layer comprising atorvastatin or a pharmaceutically acceptable salt thereof and magnesium carbonate in a weight ratio of 1:4 to 1:5

In accordance with another aspect of the present invention, there is provided a method for preparing the bilayered composite tablet formulation, comprising:

1) forming granules comprising irbesartan or a pharmaceutically acceptable salt thereof;

2) forming granules comprising atorvastatin or a pharmaceutically acceptable salt thereof, and magnesium carbonate in a weight ratio of 1:4 to 1:5; and

3) compressing the irbesartan granules formed in step 1) and the atorvastatin granules formed in step 2) into a bilayered tablet.

### BRIEF DESCRIPTION OF THE DRAWINGS

The above and other objects and features of the present invention will become apparent from the following description of the invention, when taken in conjunction with the accompanying drawings, which respectively show:

FIG. 1: a comparison of dissolution profile between irbesartan-atorvastatin bilayered tablets prepared in Comparative Examples 1 to 3 and Examples 1 to 3, and the commercially available product (Lipitor Tab.).

FIG. 2: a comparison of 10-min dissolution rate of atorvastatin between irbesartan-atorvastatin bilayered tablets prepared in Comparative Examples 1 to 3 and Examples 1 to 3, and the commercially available product (Lipitor Tab.).

FIG. 3: a comparison of blood atorvastatin level-time profile between irbesartan-atorvastatin bilayered tablets prepared in Comparative Examples 1 to 3 and Examples 1 to 3, and the commercially available product (Lipitor Tab.).

### DETAILED DESCRIPTION OF THE INVENTION

The present invention provides a bilayered composite tablet formulation comprising: (a) a first layer comprising irbesartan or a pharmaceutically acceptable salt thereof; and (b) a second layer comprising atorvastatin or a pharmaceutically acceptable salt thereof and magnesium carbonate in a weight ratio of 1:4 to 1:5.

A detailed description will be given of properties and types of ingredients used in the bilayered composite tablet formulation of the present invention below.

(i) First Layer

The first layer of the bilayered composite tablet formulation according to the present invention comprises irbesartan or a pharmaceutically acceptable salt thereof.

Irbesartan (IUPAC name: 2-butyl-3-({4-[2-(2H-1,2,3,4-tetrazol-5-yl)phenyl]phenyl}methyl)-1,3-diazaspiro[4.4]non-1-en-4-one) is a long-acting angiotensin II-receptor antagonist having high specificity with an angiotensin receptor. Irbesartan functions to block activities of angiotensin including vasoconstriction, the release of aldosterone, and the reabsorption of water and sodium, and therefore, irbesartan is applicable to the treatment of cardiovascular diseases, such as hypertension and heart failure, inter alia. Irbesartan has the structure of the formula (II) below, and is disclosed in U.S. Pat. No. 5,270,317.

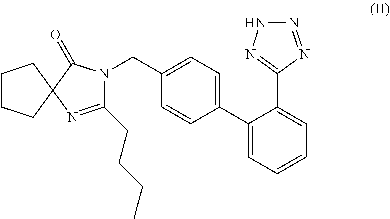 [SMILES: CCCCC1=NC2(CCCC2)C(=O)N1Cc1ccc(-c2ccccc2-c2nn[nH]n2)cc1]

In the present invention, any pharmaceutically acceptable salt of irbesartan may be used so long as it is readily available to those skilled in the art, including hydrochlorides, sodium salts, potassium salts, magnesium salts, and ammonium salts.

In the first layer according to the present invention, irbesartan or a pharmaceutically acceptable salt thereof may be contained in an amount of from 20 to 80 wt %, based on the total weight of the first layer, and preferably in an amount of from 50 to 70 wt %, with correspondence to a therapeutically effective amount ranging from 8 to 600 mg and preferably from 100 to 200 mg in each unit formulation form, but not limited thereto.

In addition, the first layer may further comprise a pharmaceutically acceptable additive. The pharmaceutically acceptable additive may be selected from the group consisting of an aqueous diluent, a binder, a disintegrant, a lubricant, a surfactant and a mixture thereof.

In the present invention, the aqueous diluent may be selected from the group consisting of microcrystalline cellulose, hydroxypropyl cellulose, pre-gelatinized starch, glucose, sucrose, lactose, sorbitol, mannitol, dulcitol, ribitol, xylitol, and a mixture thereof, but not limited thereto. The aqueous diluent may be used in an amount of from 5 to 50 wt %, based on the total weight of the first layer, and preferably in an amount of from 8 to 30 wt %.

In the present invention, the binder may be selected from the group consisting of alginic acid, sodium alginate, sodium carboxymethyl cellulose, ethyl cellulose, hydroxyethyl cellulose, hydroxypropyl cellulose, hydroxymethyl cellulose, methyl cellulose, gelatin, povidone, starch, pre-gelatinized starch, and a mixture thereof, but not limited thereto. The binder may be used in an amount of from 0.5 to 10 wt %, based on the total weight of the first layer, and preferably in an amount of from 2 to 5 wt %.

The disintegrant in the present invention may be selected from the group consisting of alginic acid, sodium alginate, sodium carboxymethyl cellulose, microcrystalline cellulose, powdered cellulose, croscarmellose sodium, crospovidone, pre-gelatinized starch, sodium glycolate, starch, and a mixture thereof, but not limited thereto. The disintegrant may be used in an amount of from 0.5 to 20 wt %, based on the total weight of the first layer, and preferably in an amount of from 2 to 10 wt %.

In the present invention, the lubricant may be selected from the group consisting of, but not limited to, calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, sodium benzoate, talc, and a mixture thereof. The lubricant may be used in an amount of from 0.1 to 10 wt %, based on the total weight of the first layer, and preferably in an amount of from 0.5 to 2 wt %.

In the present invention, the surfactant may be selected from the group consisting of sodium lauryl sulfate, poloxamer, polyethylene glycol, and a mixture thereof, but not limited thereto. The surfactant may be used in an amount of from 0.5 to 20 wt %, based on the total weight of the first layer, and preferably in an amount of from 2 to 5 wt %.

(ii) Second Layer

In the second layer of the bilayered composite tablet formulation according to the present invention, atorvastatin or a pharmaceutically acceptable salt thereof is mixed with magnesium carbonate in a weight ratio of from 1:4 to 1:5.

Atorvastatin functions to lower blood lipoprotein or lipid levels, and is used for the prevention or treatment of hyperlipidemia and arteriosclerosis.

Any pharmaceutically acceptable salt of atorvastatin that is readily available to those skilled in the art may be used in the present invention, as represented by calcium salts, hydrochlorides, sodium salts, potassium salts, magnesium salts, and ammonium salts, with preference for calcium salts. Preferably, atorvastatin is in an anhydride form.

In the second layer according to the present invention, atorvastatin or a pharmaceutically acceptable salt thereof is comprised in an amount of from 0.5 to 20 wt %, based on the total weight of the second layer, and preferably in an amount of from 2 to 5 wt %, with correspondence to a therapeutically effective amount ranging from 1 to 80 mg and preferably from 5 to 50 mg in each unit formulation form, but not limited thereto.

Existing only in the same layer that contains atorvastatin, magnesium carbonate enhances not only the stability of atorvastatin with the concomitant increase of atorvastatin in dissolution rate and bioavailability, but also prevents the reaction of atorvastatin with irbesartan in accordance with the present invention.

As mentioned above, the weight ratio of atorvastatin to magnesium carbonate in the second layer ranges from 1:4 to 1:5. If the weight ratio of atorvastatin to magnesium carbonate is less than 1:4, a higher dose of atorvastatin is required to achieve a therapeutically effective level or a desired therapeutic effect. On the other hand, when the weight ratio of atorvastatin to magnesium carbonate exceeds 1:5, no additional increments of the dissolution profile or blood concentration are observed. In addition, excess magnesium carbonate is disadvantageous in terms of drug compliance and production efficiency. Moreover, when the weight ratio departs from the range, the Cₘₐₓ of the solid dosage form is beyond 125% of that of a pre-existing marketed agent (e.g., Lipitor Tab.), which may lead to regarding the formulation as a different drug from pre-existing, commercially available ones. If so, a report on safety profiles obtained by conducting additional experiments must be submitted. Accordingly, the weight ratio of atorvastatin to magnesium carbonate in the present invention is preferably in a range of 1:4 to 1:5.

In addition, the second layer may further comprise a pharmaceutically acceptable additive. The pharmaceutically acceptable additive may be selected from the group consisting of an aqueous diluent, a binder, a disintegrant, a lubricant, and a mixture thereof.

In the present invention, the aqueous diluent may be selected from the group consisting of microcrystalline cellulose, hydroxypropyl cellulose, pre-gelatinized starch, glucose, sucrose, lactose, sorbitol, mannitol, dulcitol, ribitol, xylitol, and a mixture thereof, but not limited thereto. The aqueous diluent may be used in an amount of from 5 to 80 wt %, based on the total weight of the second layer, and preferably in an amount of from 10 to 50 wt %.

In the present invention, the binder may be selected from the group consisting of, but not limited to, alginic acid, sodium alginate, sodium carboxymethyl cellulose, ethyl cellulose, hydroxyethyl cellulose, hydroxypropyl cellulose, hydroxymethyl cellulose, methyl cellulose, gelatin, povidone, starch, pre-gelatinized starch, and a mixture thereof. The binder may be used in an amount of from 0.1 to 5 wt %, based on the total weight of the second layer, and in an amount of from 0.5 to 2 wt %.

The disintegrant in the present invention may be selected from the group consisting of alginic acid, sodium alginate, sodium carboxymethyl cellulose, microcrystalline cellulose, powdered cellulose, croscarmellose sodium, crospovidone, pre-gelatinized starch, sodium glycolate, starch, and a mixture thereof, but not limited thereto. The disintegrant may be used in an amount of from 2 to 50 wt %, based on the total weight of the second layer, and preferably in an amount of from 5 to 20 wt %.

The lubricant in the present invention may be selected from the group consisting of, but not limited to, calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, sodium benzoate, talc, and a mixture thereof. The lubricant may be used in an amount of from 0.1 to 5 wt %, based on the total weight of the second layer, and preferably in an amount of from 0.5 to 2 wt %.

(iii) Bilayered Tablet

The bilayered composite tablet formulation according to the present invention has a bilayer structure in which granules of irbesartan or a pharmaceutically acceptable salt thereof are present in a first layer while granules of a mixture of atorvastatin or a pharmaceutically acceptable salt thereof and magnesium carbonate in a weight ratio of 1:4 to 1:5 form a second layer, whereby the two granules can be blocked from contacting each other as much as possible while improving atorvastatin in dissolution rate and bioavailability.

Particularly, the bilayered composite tablet formulation according to the present invention characterized by the coexistence of atorvastatin and magnesium carbonate in a weight ratio of 1:4 to 1:5 in the second layer can avoid the problems of stability and dissolution rate, which are the most important factors in the pharmacokinetics of composite drug forms.

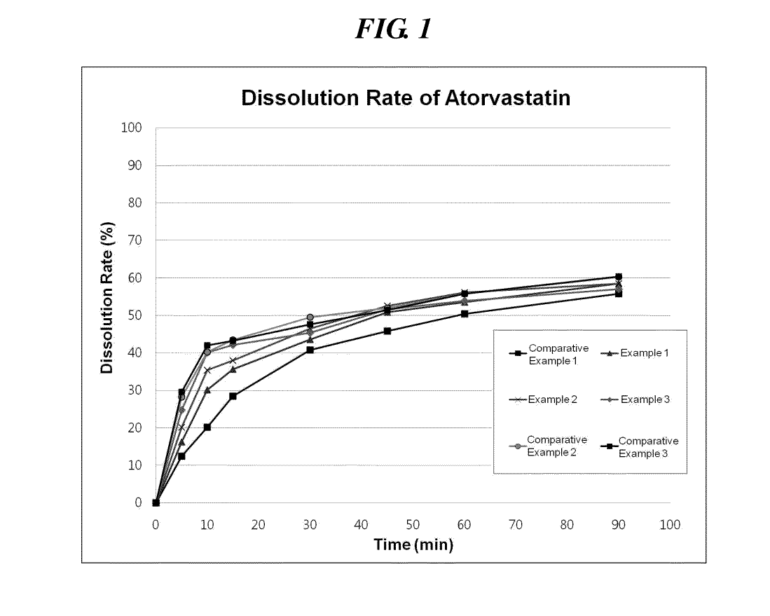

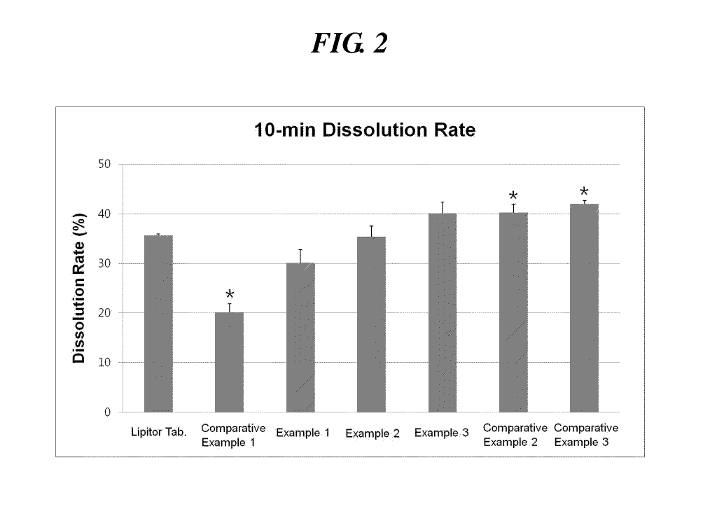

The bilayered composite tablet formulation according to one embodiment of the present invention can release atorvastatin at a rate of 30% or greater within 10 min, which is the most discriminative time range on the dissolution profile (see FIGS. 1 and 2), which is similar or superior to the commercially available product Lipitor Tab. in terms of pharmaceutical efficacy.

The bilayered composite tablet formulation of the present invention can be prepared using a method, which comprises the steps of: 1) forming granules comprising irbesartan or a pharmaceutically acceptable salt thereof; 2) forming granules comprising atorvastatin or a pharmaceutically acceptable salt thereof, and magnesium carbonate in a weight ratio of 1:4 to 1:5; and 3) compressing the irbesartan granules formed in step 1) and the atorvastatin granules formed in step 2) into a bilayered tablet using a bilayer tablet press.

The steps of the preparation method of the present invention may be carried out using conventional processes.

In one embodiment of the present invention, the granules of irbesartan or atorvastatin may be formed according to the granulation process, which comprises the steps of:

(a) blending irbesartan or atorvastatin with a disintegrant and optionally a part or all of the other additives necessary for the final composition (the additives may include a diluent, a binder and other agents necessary for processability, fluidity, stability, and formation of the unit dosage form);

(b) adding a granulation solvent to the blend obtained in step (a) (a preferred granulation solvent may be water, ethanol, isopropyl alcohol, or a mixture thereof, and other ingredients known in the art (e.g., a binder, a wetting agent, a surfactant, etc.) may be added to the granulation solvent);

(c) drying the resulting damp mass obtained in step (b) using an air dryer, a tray-dryer, a fluid-bed dryer or a microwave drier (the drying process may be performed at, e.g., 40 to 60° C.);

(d) crushing or sieving the dried matter obtained in step (c) (using a sieve with a 14 to 40 mesh, e.g., 30 mesh); and

(e) mixing the powder obtained in step (d) with an additional agent (e.g., a lubricant) and forming the mixture into granules.

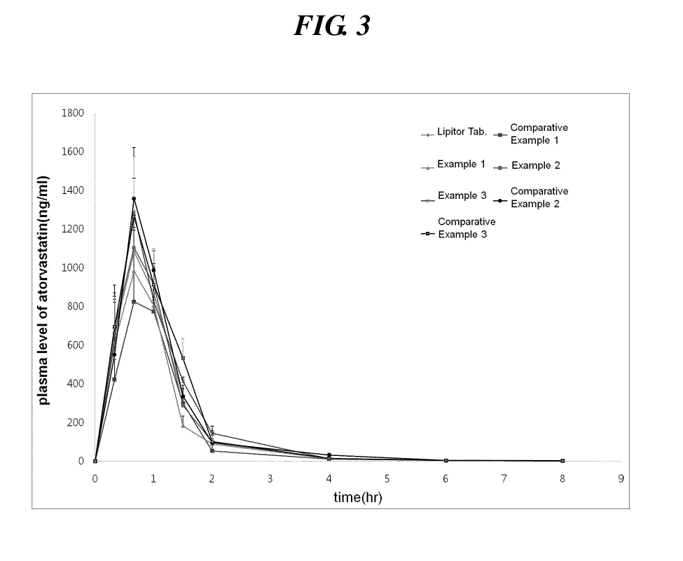

If the AUC or Cₘₐₓ of a new composite formulation exceeds 125% of that of a pre-existing commercially available drug formulation, the new composite formulation is regarded as being different in stability profile from the commercially available drug formulation. In this case, a report on the stability profile of the composite formulation must be submitted for acquiring official approval of permission. However, an experiment for providing data relevant to toxicity of the formulation is inefficient in light of temporal and financial aspects. Further, even if an additional clinical trial is performed for a new composite formulation, it does not guarantee the safety of the composite formulation. Accordingly, AUC or Cₘₐₓ, among others, is an important factor to be considered in developing a composite formulation. The bilayered composite tablet formulation of the present invention in which irbesartan and atorvastatin are confined within first and second layers, respectively, ranges in AUC T/R ratio from 0.85 to 1.18, with Cₘₐₓ values identical to that of the commercially available product (Lipitor Tab.) on a 90% confidence interval (see FIG. 3 and Table 5), and therefore exhibits a safety profile equal to that of the commercially available product.

Accordingly, the composite formulation of the present invention with superior drug uptake in the body and dissolution properties is useful as a therapeutic for hypertension and hypercholesterolemia.

### EXAMPLES

Hereinafter, the present invention is described in more detail. The following Examples are given for the purpose of illustration only, and are not intended to limit the scope of the invention.

### Preparation Example 1

### Preparation of Irbesartan Granules

As indicated by the composition of Table 1, irbesartan (Hanmi Fine Chemical, Korea) was blended with mannitol, pre-gelatinized starch and croscarmellose sodium (DMV International), added with a liquid binder of povidone (BASF, Germany) and Poloxamer 188 (BASF, Germany) in water, and dried, followed by screening the damp mass through a 30-mesh sieve to obtain wet granules. Subsequently, the wet granules were mixed with magnesium stearate to prepare irbesartan granules.

TABLE 1
Preparation of Irbesartan Granules

| Ingredient (unit: mg) | Preparation Example 1 |
|---|---|
| Irbesartan | 150 |
| Mannitol | 47 |
| Pre-gelatinized starch | 23 |
| Croscarmellose sodium | 12 |
| Povidone | 8 |
| Poloxamer188 | 9 |
| <Water> | <80> |
| Magnesium stearate | 4 |
| Total | 253 |

### Preparation Examples 2-1 to 2-6

### Preparation of Atorvastatin Granules

According to the data given in Table 2, atorvastatin calcium (TEVA, India) was blended with lactose, microcrystalline cellulose, crospovidone (BASF, Germany) and magnesium carbonate (Tomita, Japan), added with a liquid binder of HPC (Nippon Soda, Japan) and polysorbate 80 (Croda, USA), and dried, followed by screening the damp mass through a 30-mesh sieve to obtain wet granules. Subsequently, the wet granules were mixed with magnesium stearate to prepare atorvastatin granules.

TABLE 2
Preparation of Atorvastatin Granules

| | Preparation Examples | | | | | |
|---|---|---|---|---|---|---|
| Ingredient (unit: mg) | 2-1 | 2-2 | 2-3 | 2-4 | 2-5 | 2-6 |
| Atorvastatin calcium | 10.85 | 10.85 | 10.85 | 10.85 | 10.85 | 10.85 |
| (Atorvastatin itself) | (10) | (10) | (10) | (10) | (10) | (10) |
| Lactose | 120 | 120 | 120 | 120 | 120 | 120 |
| Microcrystalline cellulose | 65.6 | 65.6 | 65.6 | 65.6 | 65.6 | 65.6 |
| Crospovidone | 36 | 36 | 36 | 36 | 36 | 36 |
| Magnesium carbonate | 35 | 40 | 45 | 50 | 57 | 66 |
| (Wt ratio to Atorvastatin) | 3.5 | 4 | 4.5 | 5 | 5.7 | 6.6 |
| HPC | 3 | 3 | 3 | 3 | 3 | 3 |
| Polysorbate 80 | 1.2 | 1.2 | 1.2 | 1.2 | 1.2 | 1.2 |
| <Water> | <300> | <300> | <300> | <300> | <300> | <300> |
| Magnesium stearate | 3 | 3 | 3 | 3 | 3 | 3 |
| Total | 274.65 | 279.65 | 284.65 | 289.65 | 296.65 | 305.65 |

### Comparative Examples 1 to 3

### Preparation of Irbesartan-Atorvastatin Bilayered Tablet

As shown in Table 3 below, granules prepared in Preparation Example 1, and Preparation Examples 2-1, 2-5 or 2-6, were combined to prepare composite formulations comprising irbesartan and atorvastatin.

Using a bilayer tablet press, irbesartan granules and atorvastatin granules were compressed into bilayered tablets consisting of a first layer comprising 150 mg of irbesartan and a second layer comprising 10 mg of atorvastatin per tablet.

### Examples 1 to 3

### Preparation of Irbesartan-Atorvastatin Bilayered Tablet

As shown in Table 3 below, granules prepared in Preparation Example 1, and Preparation Examples 2-2, 2-3 or 2-4, were combined to prepare composite formulations comprising irbesartan and atorvastatin.

Using a bilayer tablet press, irbesartan granules and atorvastatin granules were compressed into bilayered tablets consisting of a first layer comprising 150 mg of irbesartan and a second layer comprising 10 mg of atorvastatin per tablet.

TABLE 3
Preparation of Composite Formulations Comprising Irbesartan and
Atorvastatin

| | Tablet | Irbesartan | Atorvastatin |
|---|---|---|---|
| Comparative | Bilayered | Preparation Example | Preparation Example |
| Example 1 | Tablet | 1 | 2-1 |
| Example 1 | Bilayered | Preparation Example | Preparation Example |
| | Tablet | 1 | 2-2 |
| Example 2 | Bilayered | Preparation Example | Preparation Example |
| | Tablet | 1 | 2-3 |
| Example 3 | Bilayered | Preparation Example | Preparation Example |
| | Tablet | 1 | 2-4 |
| Comparative | Bilayered | Preparation Example | Preparation Example |
| Example 2 | Tablet | 1 | 2-5 |
| Comparative | Bilayered | Preparation Example | Preparation Example |
| Example 3 | Tablet | 1 | 2-6 |

### Experimental Example 1

### Dissolution Assay of Composite Formulation

The bilayered tablets prepared in Comparative Examples 1 to 3, and Examples 1 to 3, were assayed for the dissolution of atovastatin. According to USP dissolution apparatus 2 (Paddle method), a dissolution test was performed using 900 mL of a dissolution liquid, pH 1.2, while stirring at 25 rpm. Samples were taken at 5, 10, 15, 30, 45, 60 and 90 min after the dissolution, and measured for the dissolution rate of atorvastatin. The results are shown in Table 1. As shown in FIG. 1, the dissolution rate of atorvastatin was observed to increase with an increase in magnesium carbonate content.

In addition, data of the dissolution rate of the bilayered tablets prepared in Comparative Examples 1 to 3 and Examples 1 to 3 are shown in FIG. 2, and p values of the t-test are listed in Table 4 below. For comparison, the commercially available product Lipitor Tab. was employed as a control.

As is understood from the data, all the composite formulations of Examples 1 to 3 were found to be substantially equal to the control in dissolution rate, with a significance of 5%, considering the dissolution rates similar to that of the control, at time points within 10 min, the most distinctive time range, with p values ≧0.05. On the other hand, all of the composite formulations of Comparative Examples 1 to 3 were shown to significantly differ from the control, with p values <0.05. In FIG. 2, formulations with significant differences are indicated by an asterisk (*).

TABLE 4
T Test Result - P Value

| | Comparative | | | | Comparative | Comparative |
|---|---|---|---|---|---|---|
| | Example 1 | Example 1 | Example 2 | Example 3 | Example 2 | Example 3 |
| P value | 0.006 | 0.083 | 0.900 | 0.059 | 0.038 | 0.005 |

### Experimental Example 2

### Assay for Bioavailability of Atorvastatin

To evaluate the bioavailability of atorvastatin of the bilayered tablets prepared in Comparative Examples 1 to 3 and Examples 1 to 3, the prepared formulations were administered to beagle dogs to monitor blood atorvastatin levels. For comparison, the commercially available product Lipitor Tab. (Pfizer) was used as a control.

A total of 28 beagle dogs were divided into seven groups of four. The tablets were crushed and dispersed in 0.5% carboxymethyl cellulose (CMC), and orally administered at a dose corresponding to 10 mg/kg atorvastatin to the beagle dogs. Blood atorvastatin levels were measured by time after administration, and analyzed for pharmacokinetic parameters of atorvastatin. The results are shown in FIG. 3 and Table 5.

TABLE 5
Pharmacokinetic Parameters of Atorvastatin

| | | Comparative | | | | Comparative | Comparative |
|---|---|---|---|---|---|---|---|
| | Control | Example 1 | Example 1 | Example 2 | Example 3 | Example 2 | Example 3 |
| AUC | 1345.1 | 962.3 | 1144.8 | 1351.6 | 1587.6 | 1724.5 | 1748.8 |
| (ng · hr/mL) | | | | | | | |
| Cₘₐₓ | 1083.2 | 824.1 | 982.5 | 1102.6 | 1285.5 | 1357.2 | 1264.2 |
| (ng/mL) | | | | | | | |
| Tₘₐₓ (hr) | 0.8 | 0.7 | 0.6 | 0.8 | 0.7 | 0.7 | 0.7 |
| AUC T/R | — | 0.715 | 0.851 | 1.005 | 1.180 | 1.282 | 1.300 |
| Ratio | | | | | | | |

As is apparent from the data of FIG. 3 and Table 5, the composite formulations of Examples 1 to 3 were found to range in AUC T/R ratio from 0.85 to 1.18, with Cₘₐₓ values identical to that of the commercially available product (Lipitor Tab.) on a 90% confidence interval.

What is claimed is:

1. A bilayered composite tablet formulation, comprising:
(a) a first layer comprising irbesartan or a pharmaceutically acceptable salt thereof; and
(b) a second layer comprising atorvastatin or a pharmaceutically acceptable salt thereof and magnesium carbonate in a weight ratio of 1:4 to 1:5.

2. The bilayered composite tablet formulation of claim 1, wherein the atorvastatin is in an anhydride form.

3. The bilayered composite tablet formulation of claim 1, wherein the irbesartan or the pharmaceutically acceptable salt thereof is contained in an amount of from 8 mg to 600 mg per unit formulation.

4. The bilayered composite tablet formulation of claim 1, wherein the atorvastatin or the pharmaceutically acceptable salt thereof is contained in an amount of from 1 mg to 80 mg per unit formulation.

5. The bilayered composite tablet formulation of claim 1, wherein the first layer further comprises a pharmaceutically acceptable additive selected from the group consisting of an aqueous diluent, a binder, a disintegrant, a lubricant, a surfactant, and a mixture thereof.

6. The bilayered composite tablet formulation of claim 1, wherein the second layer further comprises a pharmaceutically acceptable additive selected from the group consisting of an aqueous diluent, a binder, a disintegrant, a lubricant, and a mixture thereof.

7. A method for preparing the bilayered composite tablet formulation of claim 1, which comprises the steps of:
1) forming granules comprising irbesartan or a pharmaceutically acceptable salt thereof;
2) forming granules comprising atorvastatin or a pharmaceutically acceptable salt thereof, and magnesium carbonate in a weight ratio of 1:4 to 1:5; and
3) compressing the irbesartan granules formed in step 1) and the atorvastatin granules formed in step 2) into a bilayered tablet.
